(19) United States
(12) Patent Application Publication
(10) Pub. No.: US 2013/0237745 A1
(43) Pub. Date: Sep. 12, 2013

(54) ENVIRONMENT CONTROL APPARATUS, ENVIRONMENT CONTROL METHOD, AND PROGRAM

(71) Applicant: SONY CORPORATION, Tokyo (JP)

(72) Inventors: Kouichirou Ono, Tokyo (JP); Yoichiro Sako, Tokyo (JP)

(73) Assignee: Sony Corporation, Tokyo (JP)

(21) Appl. No.: 13/769,936

(22) Filed: Feb. 19, 2013

(30) Foreign Application Priority Data
Mar. 6, 2012 (JP) 2012-049080

(51) Int. Cl. A61M 21/02

(52) U.S. Cl. CPC A61M 21/02
USPC 600/28; 600/27

(57) ABSTRACT

Provided is an environment control apparatus including an input unit into which information from a medical device is input, and an environment control unit configured to control an environment perceived by a subject based on the information input into the input unit.

### BACKGROUND

The present disclosure relates to an environment control apparatus, an environment control method, and a program.

In a medical facility such as a hospital or a diagnostic laboratory, treatments and examinations that use medical devices are widely performed. For example, in ultrasound diagnosis, an echo examination that displays the internal state of the body as an image is performed by pressing an ultrasound probe against the body. Further, drug injection or blood collection are performed by inserting a syringe needle into the body. In addition, blood pressure and body temperature are routinely measured using a medical device such as a blood-pressure gauge or a thermometer.

On the other hand, research into odor production apparatuses that artificially produce an odor is also proceeding. For example, JP 2011-166430A discloses an apparatus for producing an odor that is set for an image when the image is displayed. Further, JP 2011-184486 also discloses an odor production apparatus that is capable of providing an odor.

### SUMMARY

During treatment or diagnosis at a medical facility like that described above, the subject of the treatment or diagnosis may be nervous, which can make it difficult to carry out the treatment or diagnosis well. Consequently, during the treatment or diagnosis, it is effective to relax the subject by changing an environment perceived by the subject, such as by producing an odor.

According to an embodiment of the present disclosure, there is provided a device which includes a novel and improved environment control apparatus, environment control method, and program, that are capable of alleviating a subject's nervousness during treatment and diagnosis.

According to an embodiment of the present disclosure, there is provided an environment control apparatus including an input unit into which information from a medical device is input, and an environment control unit configured to control an environment perceived by a subject based on the information input into the input unit.

Further, according to an embodiment of the present disclosure, there is provided an environment control method including receiving information from a medical device, and controlling an environment perceived by a subject based on the information.

Further according to an embodiment of the present disclosure, there is provided a program for causing a computer to function as an input unit into which information from a medical device is input and an environment control unit configured to control an environment perceived by a subject based on the information input into the input unit.

According to the embodiments of the present disclosure described above, a patient's nervousness during treatment and diagnosis can be alleviated.

### BRIEF DESCRIPTION OF THE DRAWINGS

FIG. 1 is an explanatory diagram illustrating a configuration of an odor production system according to a first embodiment of the present disclosure;

FIG. 2 is a function block diagram illustrating a configuration of an ultrasound probe and an ultrasound diagnostic apparatus according to a first embodiment of the present disclosure;

FIG. 3 is a flowchart illustrating operation of an ultrasound diagnostic apparatus according to a first embodiment of the present disclosure;

FIG. 4 is a function block diagram illustrating a configuration of an ultrasound probe and an ultrasound diagnostic apparatus according to a first modified example;

FIG. 5 is a flowchart illustrating operation according to a first modified example;

FIG. 6 is a function block diagram illustrating a configuration of an ultrasound probe and an ultrasound diagnostic apparatus according to a second modified example;

FIG. 7 is a flowchart illustrating operation according to a second modified example;

FIG. 8 is a function block diagram illustrating a configuration according to a third modified example;

FIG. 9 is a flowchart illustrating operation according to a third modified example;

FIG. 10 is a function block diagram illustrating a configuration according to a fourth modified example;

FIG. 11 is a flowchart illustrating operation according to a fourth modified example;

FIG. 12 is a function block diagram illustrating a configuration according to a fifth modified example;

FIG. 13 is a flowchart illustrating operation according to a fifth modified example;

FIG. 14 is a function block diagram illustrating a configuration according to a sixth modified example;

FIG. 15 is a flowchart illustrating operation according to a sixth modified example;

FIG. 16 is a flowchart illustrating operation according to a sixth modified example;

FIG. 17 is a flowchart illustrating operation according to a seventh modified example;

FIG. 18 is an explanatory diagram illustrating a configuration according to a second embodiment of the present disclosure; and

FIG. 19 is an explanatory diagram illustrating a configuration according to a third embodiment of the present disclosure.

### DETAILED DESCRIPTION OF THE EMBODIMENT(S)

Hereinafter, preferred embodiments of the present disclosure will be described in detail with reference to the appended drawings. Note that, in this specification and the appended drawings, structural elements that have substantially the same function and structure are denoted with the same reference numerals, and repeated explanation of these structural elements is omitted.

Further, in the present specification and drawings, a plurality of constituent elements having essentially the same function and configuration are distinguished by denoting them with a different letter of the alphabet after the same reference numeral. However, in cases where it is not particularly necessary to distinguish the plurality of constituent elements having essentially the same function and configuration, such elements are denoted with the same reference numeral.

The present disclosure will be described based on the following item order.
1. Outline of the present disclosure
2. First embodiment
2-1. Configuration of the first embodiment
2-2. Configuration of an ultrasound probe and an ultrasound diagnostic apparatus
2-3. Operation of the first embodiment
3. Modified examples of the first embodiment
3-1. First modified example
3-2. Second modified example
3-3. Third modified example
3-4. Fourth modified example
3-5. Fifth modified example
3-6. Sixth modified example
3-7. Seventh modified example
4. Second embodiment
5. Third embodiment
6. Summary

### 1. OUTLINE OF THE PRESENT DISCLOSURE

The technology according to the present disclosure can be worked based on various embodiments, as is described in detail in “2. First embodiment” to “5. Third embodiment” as examples. Further, the ultrasound diagnostic apparatus and the environment control apparatus according to the respective embodiments include:
A. an input unit (input/output unit 220) into which information from a medical device is input; and
B. an environment control unit (odor control unit 252) configured to control an environment that is perceived by a subject based on the information input into the information input unit.

Here, examples of the information input from a medical device include a pressure sensor detection result, a proximity sensor detection result, a body temperature measurement result, a blood pressure measurement result, a breathing measurement result, a pulse measurement result and the like. Further, examples of the environment perceived by the subject that is used by the medical device include odor, sound, video, temperature, humidity and the like.

Namely, the technology according to the present disclosure can alleviate a subject's nervousness by controlling an environment perceived by the subject based on the nervous state of the subject inferred from information that is input from a medical device like that described above. Consequently, a good medical effect, diagnostic result or the like can be obtained. The respective embodiments according to the present disclosure will now be successively described in more detail.

### 2. FIRST EMBODIMENT

A treatment or diagnosis subject is often in a nervous state due to worries about whether the treatment or examination will be painful or whether he/she has something seriously wrong with his/her health. To alleviate this nervous state, it is effective to arrange a fragrance to soothe feelings in the treatment room or examination room, for example.

However, the point when the subject is most nervous is not when he/she enters the treatment room or examination room, but rather when the treatment or examination starts. Further, it is desirable to control the odor based on the nervous state of the subject or the usage condition of the medical device. Consequently, just constantly producing the same odor by arranging a fragrance in the treatment room or examination room is not enough to obtain a sufficient effect. Accordingly, as a first embodiment, a configuration will now be described that produces an odor based on information input from a medical device.

### 2-1. System Configuration of the First Embodiment

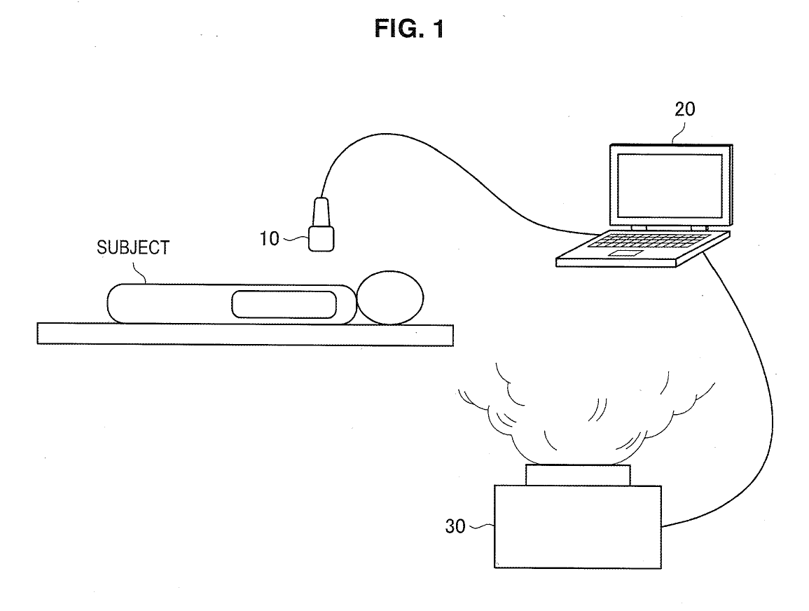

FIG. 1 is an explanatory diagram illustrating a system configuration of an odor production system according to the first embodiment. As illustrated in FIG. 1, the odor production system according to the first embodiment includes an ultrasound probe 10, an ultrasound diagnostic apparatus 20, and an odor production apparatus 30.

The ultrasound probe 10 is a medical device that transmits ultrasonic waves onto a subject and receives the reflected ultrasonic waves. The ultrasound probe 10, which is connected to the ultrasound diagnostic apparatus 20, transmits ultrasonic waves based on a control from the ultrasound diagnostic apparatus 20, and outputs the received ultrasonic waves (hereinafter, “echo signal”) to the ultrasound diagnostic apparatus 20.

The ultrasound diagnostic apparatus 20 displays a state of an examination site of the subject as a video by converting the echo signal input from the ultrasound probe 10 into image data. Further, the ultrasound diagnostic apparatus 20 also functions as an environment control apparatus for controlling the odor production apparatus 30 based on information input from a medical device, such as the ultrasound probe 10.

The odor production apparatus 30 is an example of an environment change apparatus that changes the environment perceived by the subject by producing an odor based on a control from the ultrasound diagnostic apparatus 20. Note that although FIG. 1 illustrates a case in which the odor production apparatus 30 is provided separately to the ultrasound diagnostic apparatus 20, the odor production apparatus 30 can be provided in the ultrasound diagnostic apparatus 20.

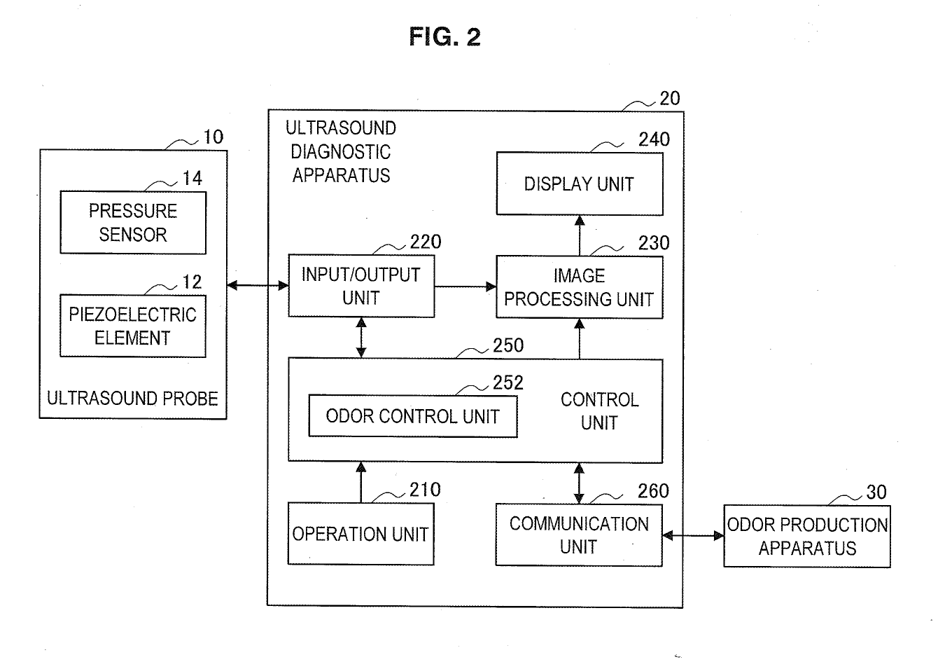

The configuration of the odor production apparatus according to the first embodiment was described above. Next, a more detailed configuration of the ultrasound probe 10 and the ultrasound diagnostic apparatus 20 will be described with reference to FIG. 2.

### 2-2. Configuration of the Ultrasound Probe 10 and the Ultrasound Diagnostic Apparatus 20

FIG. 2 is a function block diagram illustrating a configuration of the 10 and the ultrasound diagnostic apparatus 20 according to the first embodiment. As illustrated in FIG. 2, the ultrasound probe 10 includes a piezoelectric element 12 and a pressure sensor 14.

The piezoelectric element 12 is an oscillator that sends and receives ultrasonic waves based on a piezoelectric effect. The pressure sensor 14 detects pressure based on contact between the ultrasound probe 10 and another object. For example, the pressure sensor 14 may be provided on a contact face between the ultrasound probe 10 and the subject to detect the contact pressure between the ultrasound probe 10 and the subject.

Further, as illustrated in FIG. 2, the ultrasound diagnostic apparatus 20 includes an operation unit 210, an input/output unit 220, an image processing unit 230, a display unit 240, a control unit 250, and a communication unit 260.

The operation unit 210 is a unit that lets an operator, such as a doctor or a medical technologist, operate the ultrasound diagnostic apparatus 20. The operation unit 210 outputs signals to the control unit 250 based on operations made by the operator.

The input/output unit 220 functions as an input unit into which echo signals and information are input from a medical device like the ultrasound probe 10, and an output unit that outputs ultrasonic waves control signals for transmitting ultrasonic waves to the ultrasound probe 10.

The image processing unit 230 converts echo signals supplied from the input/output unit 220 into image data.

The display unit 240 displays image data indicating a state of the examination site of the subject obtained by the image processing unit 230. The display unit 240 may be a CRT (cathode ray tube) display device, a liquid crystal display (LCD) device, or an OLED organic light emitting diode) device.

The control unit 250 controls the overall operation of the ultrasound diagnostic apparatus 20. Further, the control unit 250 according to the present embodiment has a function of an odor control unit 252. The odor control unit 252 is an example of an environment control unit for controlling odor production by the odor production apparatus 30. This odor control unit 252 controls the odor production apparatus 30 based on the usage state of the ultrasound probe 10 indicated by the information input into the input/output unit 220.

The communication unit 260, which is an interface with the odor production apparatus 30, transmits control signals to the odor production apparatus 30 from the odor control unit 252. Note that the communication unit 260 and the odor production apparatus 30 may be connected via a wire, or connected wirelessly.

The control of the odor production apparatus 30 by the odor control unit 252 will now be described in more detail. As described above, a subject's nervousness can increase when a treatment or examination is performed. Consequently, producing an odor that alleviates nervousness when the ultrasound probe 10 starts being used can be thought to be effective.

Therefore, the odor control unit 252 determines whether the ultrasound probe 10 is in contact with the subject based on a pressure value of the ultrasound probe 10 detected by the pressure sensor 14. If it is determined that the ultrasound probe 10 is in contact with the subject, the odor production apparatus 30 may be made to produce an odor. Specifically, the odor control unit 252 may determine whether a pressure value satisfies a predetermined condition, such as whether the value is within an expected range during contact, and if the pressure value does satisfy the predetermined condition, make the odor production apparatus 30 produce an odor.

Based on such a configuration, an odor for alleviating nervousness can be produced at a time when a subject's nervousness is expected to increase. Consequently, a good medical effect or diagnostic effect can be obtained. Further, the odor control unit 252 can also make the odor production apparatus 30 produce an odor based on an attribute of the subject. For example, the odor control unit 252 can make the odor production apparatus 30 produce a different odor based on whether the subject is male or female, or a child or an adult. Subject attributes can be acquired based on an input operation performed by the operator or from an electronic medical record.

### 2-3. Operation of the First Embodiment

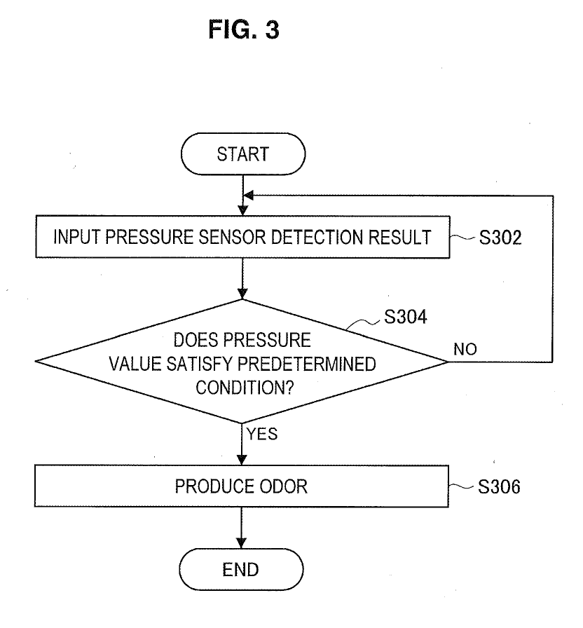

The configuration according to the first embodiment of the present disclosure was described above. Next, operation according to the first embodiment of the present disclosure will be described with reference to FIG. 3.

FIG. 3 is a flowchart illustrating operation of the ultrasound diagnostic apparatus 20 according to the first embodiment. As illustrated in FIG. 3, first, when a detection result by the pressure sensor 14 of the ultrasound probe 10 is input to the input/output unit 220 of the ultrasound diagnostic apparatus 20 (step S302), the odor control unit 252 determines whether the pressure sensor 14 detection result satisfies the predetermined condition (step S304).

If it is determined that the detection result by the pressure sensor 14 satisfies the predetermined condition, namely, that the ultrasound probe 10 is in contact with the subject, the odor control unit 252 makes the odor production apparatus 30 produce an odor (step S306). Based on this configuration, the subject's nervousness can be automatically alleviated at an appropriate timing.

### 3. MODIFIED EXAMPLES OF THE FIRST EMBODIMENT

The first embodiment of the present disclosure that was described above can be modified in various aspects. Modified examples of the first embodiment of the present disclosure will now be described below.

### 3-1. First Modified Example

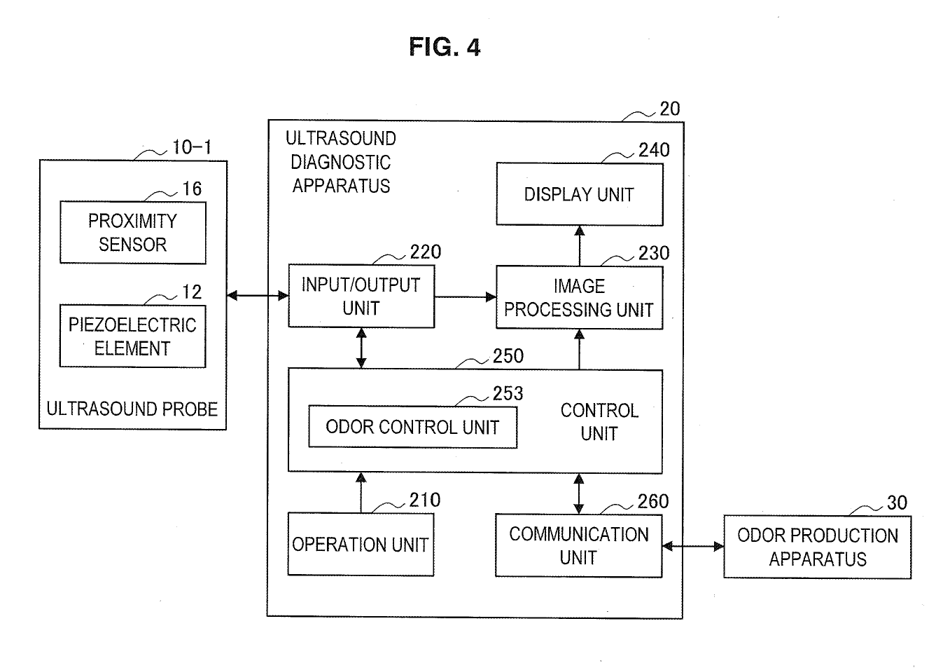

FIG. 4 is a function block diagram illustrating a configuration of an ultrasound probe 10-1 and the ultrasound diagnostic apparatus 20 according to a first modified example. As illustrated in FIG. 4, the ultrasound probe 10-1 according to the first modified example includes a piezoelectric element 12 and a proximity sensor 16.

The piezoelectric element 12 is, as described in the first embodiment, an oscillator that sends and receives ultrasonic waves based on a piezoelectric effect. The proximity sensor 16 detects whether the ultrasound probe 10 is close to another object. The proximity sensor 16 may be, for example, an infrared sensor that detects the time from transmission of infrared rays until reception of the reflected waves.

Further, as illustrated in FIG. 4, the ultrasound diagnostic apparatus 20 includes an operation unit 210, an input/output unit 220, an image processing unit 230, a display unit 240, a control unit 250, and a communication unit 260. Since these parts, except for an odor control unit 253 that is included in the control unit 250, are as described in the first embodiment, a description of those parts will be omitted here.

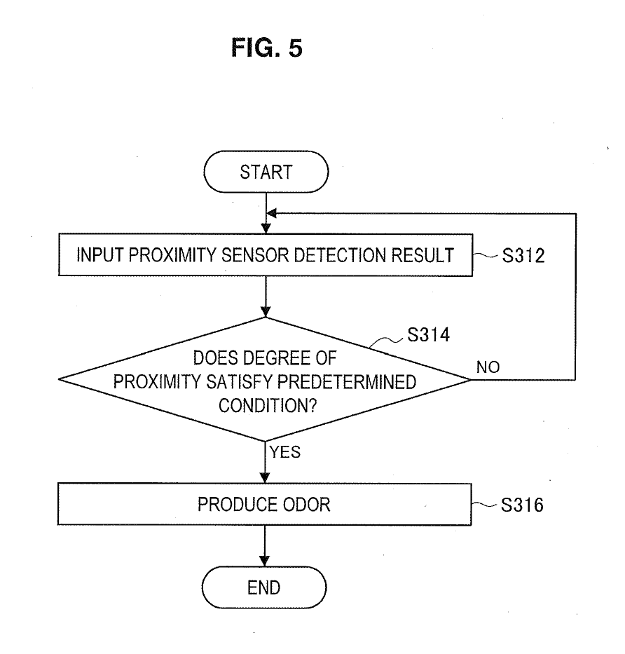

The odor control unit 253 according to the first modified example controls the odor production apparatus 30 based on the usage state of the ultrasound probe 10-1 indicated by the information input into the input/output unit 220. Specifically, as illustrated in FIG. 5, when a detection result by the proximity sensor 16 of the ultrasound probe 10-1 is input to the input/output unit 220 (step S312), the odor control unit 253 determines whether a degree of proximity between the ultrasound probe 10-1 and the subject indicated by the detection result satisfies a predetermined condition (step S314). For example, the odor control unit 253 may determine whether the ultrasound probe 10-1 and the subject are within 10 cm of each other.

If it is determined that the degree of proximity between the ultrasound probe 10-1 and the subject satisfies the predetermined condition, the odor control unit 253 makes the odor production apparatus 30 produce an odor (step S316). Based on this configuration, an odor that alleviates the subject's nervousness can be produced at a timing when the ultrasound probe 10-1 approaches the subject when use starts.

### 3-2. Second Modified Example

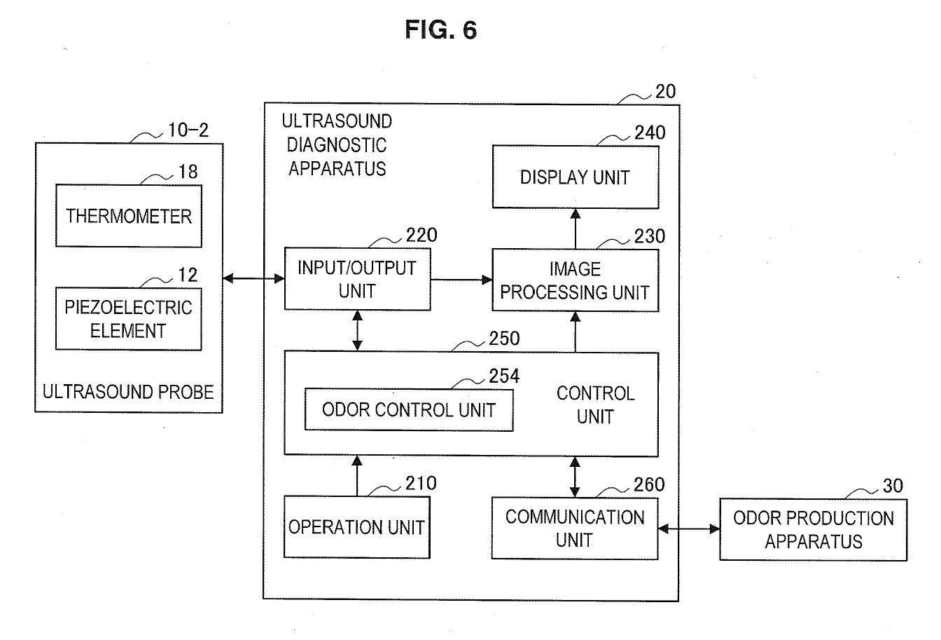

FIG. 6 is a function block diagram illustrating a configuration of an ultrasound probe 10-2 and the ultrasound diagnostic apparatus 20 according to a second modified example. As illustrated in FIG. 6, the ultrasound probe 10-2 according to the second modified example includes the piezoelectric element 12 and a thermometer 18.

The piezoelectric element 12 is, as described in the first embodiment, an oscillator that sends and receives ultrasonic waves based on a piezoelectric effect. The thermometer 18 measures the subject's body temperature.

Further, as illustrated in FIG. 6, the ultrasound diagnostic apparatus 20 includes an operation unit 210, an input/output unit 220, an image processing unit 230, a display unit 240, a control unit 250, and a communication unit 260. Since these parts, except for an odor control unit 254 that is included in the control unit 250, are as described in the first embodiment, a description of those parts will be omitted here.

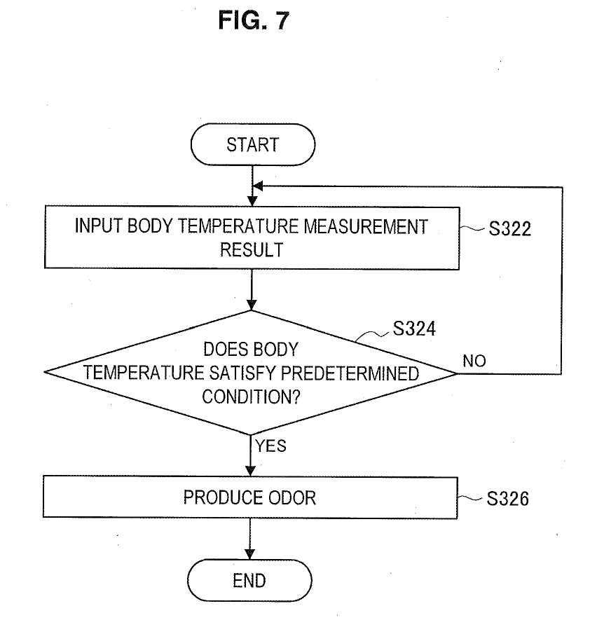

The odor control unit 254 according to the second modified example controls the odor production apparatus 30 based on the state of the subject indicated by the information input into the input/output unit 220. Specifically, as illustrated in FIG. 7, when a measurement result by the thermometer 18 of the ultrasound probe 10-2 is input to the input/output unit 220 (step S322), the odor control unit 254 determines whether the measured body temperature satisfies a predetermined condition, such as whether it indicates a body temperature exhibited during nervousness, for example (step S324). Since body temperature generally decreases when a person is nervous, the odor control unit 254 may determine whether the subject's body temperature has decreased as the predetermined condition.

If it is determined that the measurement result by the thermometer 18 satisfies the predetermined condition, the odor control unit 254 makes the odor production apparatus 30 produce an odor (step S326). Thus, according to the second modified example, the subject's nervousness can be effectively alleviated by producing an odor when the subject is thought to be nervous.

### 3-3. Third Modified Example

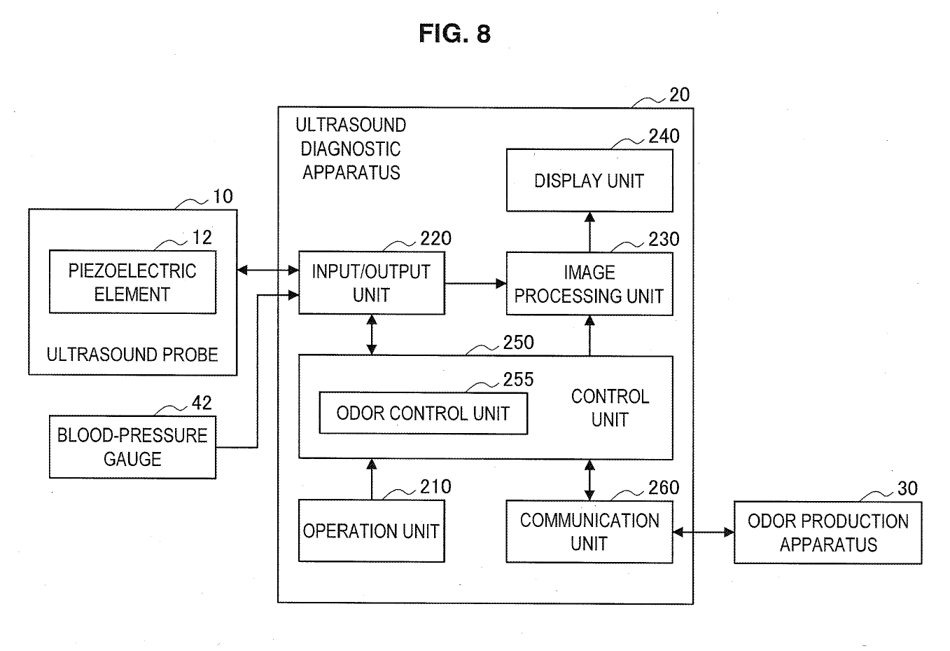

FIG. 8 is a function block diagram illustrating a configuration according to a third modified example. As illustrated in FIG. 8, the odor production system according to the third modified example includes an ultrasound probe 10, an ultrasound diagnostic apparatus 20, an odor production apparatus 30, and a blood-pressure gauge 42. The blood-pressure gauge 42 measures a subject's blood pressure, and outputs the blood pressure measurement result to the ultrasound diagnostic apparatus 20.

Further, as illustrated in FIG. 8, the ultrasound diagnostic apparatus 20 includes an operation unit 210, an input/output unit 220, an image processing unit 230, a display unit 240, a control unit 250, and a communication unit 260. Since these parts, except for an odor control unit 255 that is included in the control unit 250, are as described in the first embodiment, a description of those parts will be omitted here.

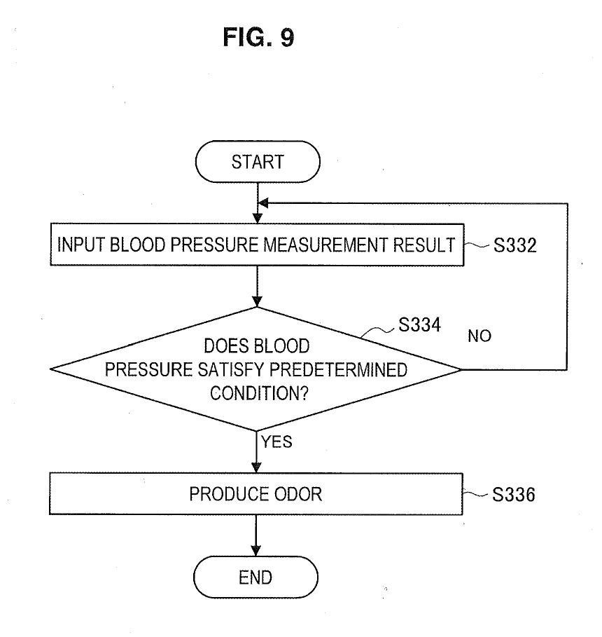

The odor control unit 255 according to the third modified example controls the odor production apparatus 30 based on the state of the subject indicated by the information input into the input/output unit 220. Specifically, as illustrated in FIG. 9, when a measurement result by the blood-pressure gauge 42 is input to the input/output unit 220 (step S332), the odor control unit 255 determines whether the measured blood pressure satisfies a predetermined condition, such as whether it indicates a blood pressure exhibited during nervousness, for example (step S334). Since blood pressure generally increases when a person is nervous, the odor control unit 255 may determine whether the subject's blood pressure has increased as the predetermined condition.

If it is determined that the measurement result by the blood-pressure gauge 42 satisfies the predetermined condition, the odor control unit 255 makes the odor production apparatus 30 produce an odor (step S336). Thus, according to the third modified example, the subject's nervousness can be effectively alleviated by producing an odor when the subject is thought to be nervous.

### 3-4. Fourth Modified Example

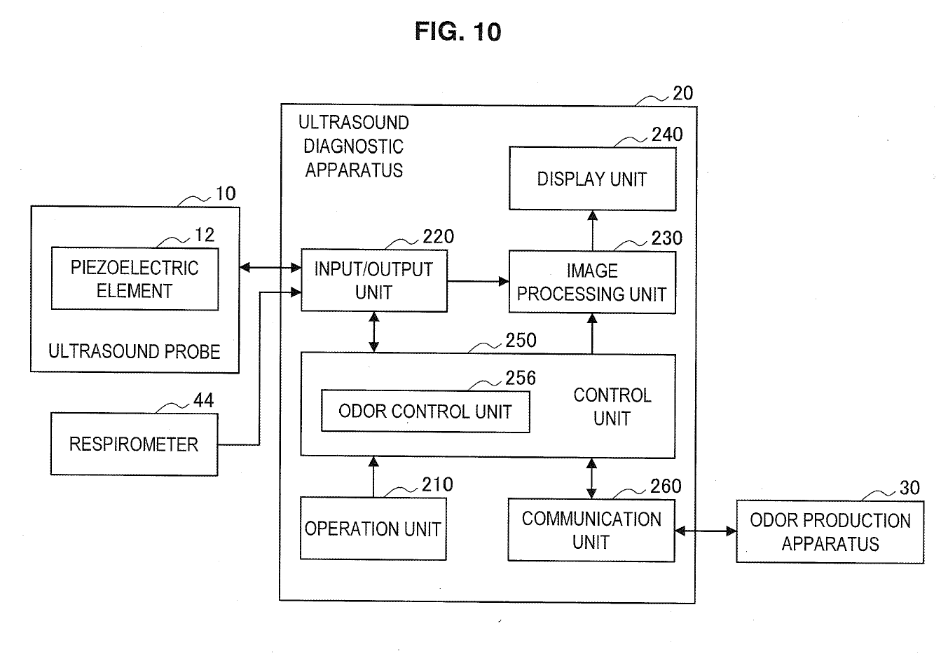

FIG. 10 is a function block diagram illustrating a configuration according to a fourth modified example. As illustrated in FIG. 10, the odor production system according to the fourth modified example includes an ultrasound probe 10, an ultrasound diagnostic apparatus 20, an odor production apparatus 30, and a respirometer 44. The respirometer 44 measures a subject's breathing cycle, and outputs the breathing cycle measurement result to the ultrasound diagnostic apparatus 20.

Further, as illustrated in FIG. 10, the ultrasound diagnostic apparatus 20 includes an operation unit 210, an input/output unit 220, an image processing unit 230, a display unit 240, a control unit 250, and a communication unit 260. Since these parts, except for an odor control unit 256 that is included in the control unit 250, are as described in the first embodiment, a description of those parts will be omitted here.

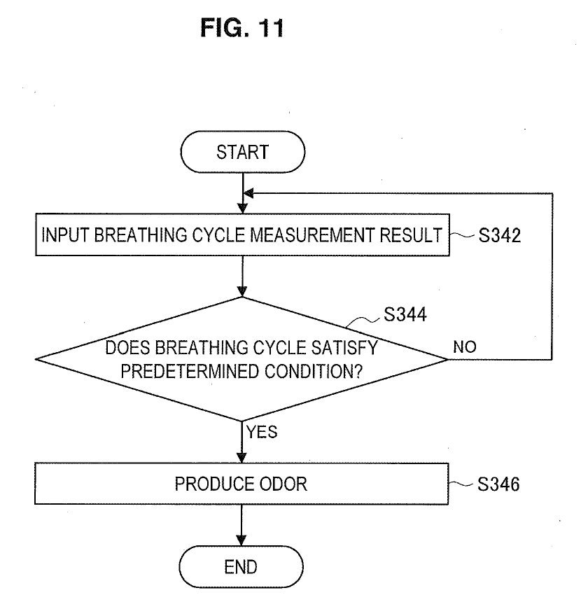

The odor control unit 256 according to the fourth modified example controls the odor production apparatus 30 based on the state of the subject indicated by the information input into the input/output unit 220. Specifically, as illustrated in FIG. 11, when a measurement result by the respirometer 44 is input to the input/output unit 220 (step S342), the odor control unit 256 determines whether the measured breathing cycle satisfies a predetermined condition, such as whether it indicates a breathing cycle that is exhibited during nervousness, for example (step S344). Since the breathing cycle generally shortens or becomes uneven when a person is nervous, the odor control unit 256 may determine whether the breathing cycle is as expected when a person is nervous or has become uneven as the predetermined condition.

If it is determined that the measurement result by the respirometer 44 satisfies the predetermined condition, the odor control unit 256 makes the odor production apparatus 30 produce an odor (step S346). Thus, according to the fourth modified example, the subject's nervousness can be effectively alleviated by producing an odor when the subject is thought to be nervous.

### 3-5. Fifth Modified Example

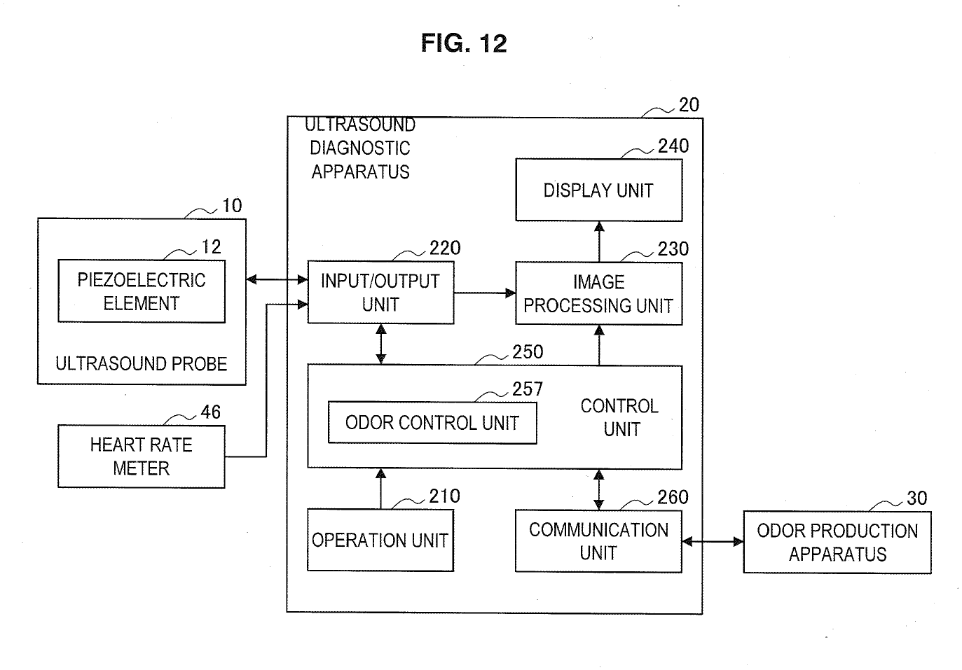

FIG. 12 is a function block diagram illustrating a configuration according to a fifth modified example. As illustrated in FIG. 11, the odor production system according to the fifth modified example includes an ultrasound probe 10, an ultrasound diagnostic apparatus 20, an odor production apparatus 30, and a heart rate meter 46. The heart rate meter 46 measures a subject's heart rate, and outputs the heart rate measurement result to the ultrasound diagnostic apparatus 20.

Further, as illustrated in FIG. 11, the ultrasound diagnostic apparatus 20 includes an operation unit 210, an input/output unit 220, an image processing unit 230, a display unit 240, a control unit 250, and a communication unit 260. Since these parts, except for an odor control unit 257 that is included in the control unit 250, are as described in the first embodiment, a description of those parts will be omitted here.

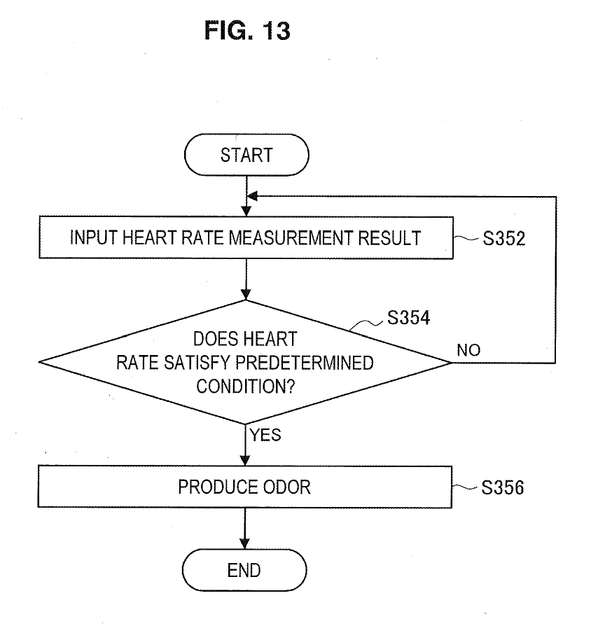

The odor control unit 257 according to the fifth modified example controls the odor production apparatus 30 based on the state of the subject indicated by the information input into the input/output unit 220. Specifically, as illustrated in FIG. 13, when a measurement result by the heart rate meter 46 is input to the input/output unit 220 (step S352), the odor control unit 257 determines whether the measured heart rate satisfies a predetermined condition, such as whether it indicates a heart rate exhibited during nervousness, for example (step S354). Since heart rate generally increases when a person is nervous, the odor control unit 257 may determine whether the heart rate is a value that would be expected during nervousness or whether the heart rate has increased as the predetermined condition.

If it is determined that the heart rate measurement result satisfies the predetermined condition, the odor control unit 257 makes the odor production apparatus 30 produce an odor (step S356). Thus, according to the fifth modified example, the subject's nervousness can be effectively alleviated by producing an odor when the subject is thought to be nervous.

### 3-6. Sixth Modified Example

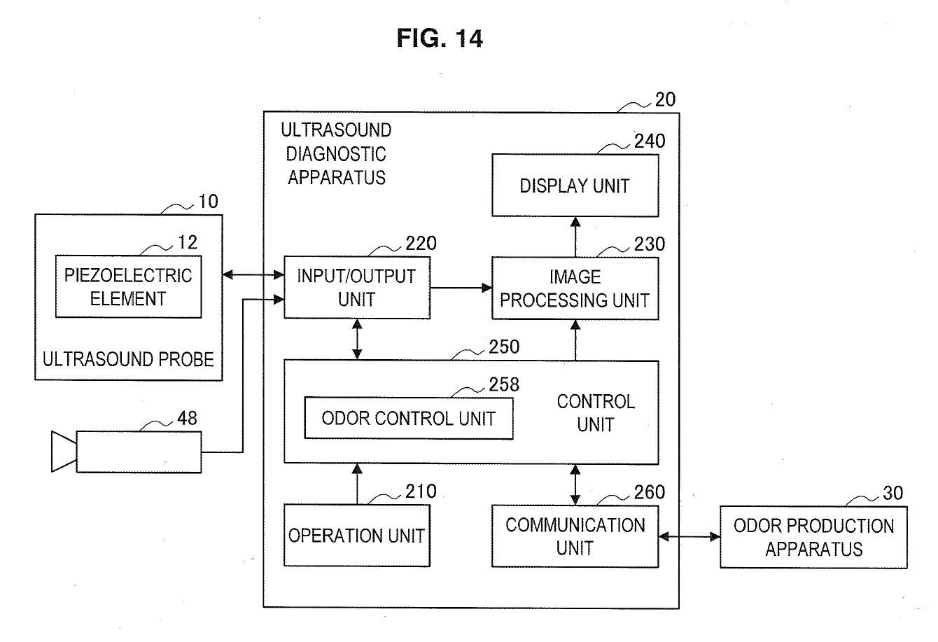

FIG. 14 is a function block diagram illustrating a configuration according to a third modified example. As illustrated in FIG. 14, the odor production system according to the sixth modified example includes an ultrasound probe 10, an ultrasound diagnostic apparatus 20, an odor production apparatus 30, and an imaging apparatus 48. The imaging apparatus 48 captures an image of the subject, and outputs the captured image of the subject obtained by imaging to the ultrasound diagnostic apparatus 20.

Further, as illustrated in FIG. 14, the ultrasound diagnostic apparatus 20 includes an operation unit 210, an input/output unit 220, an image processing unit 230, a display unit 240, a control unit 250, and a communication unit 260. Since these parts, except for an odor control unit 258 that is included in the control unit 250, are as described in the first embodiment, a description of those parts will be omitted here.

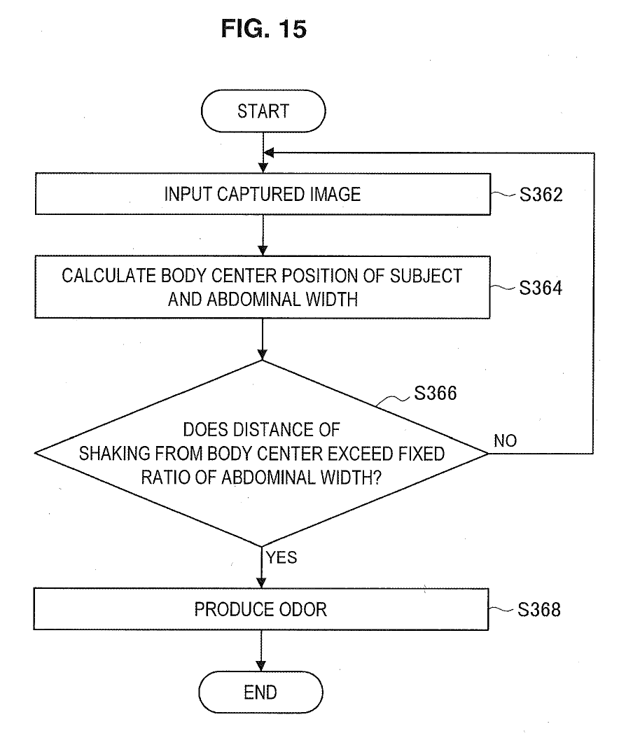

The odor control unit 258 according to the sixth modified example controls the odor production apparatus 30 based on the state of the subject indicated by the captured image input into the input/output unit 220. Specifically, as illustrated in FIG. 15, when a captured image of the subject is input to the input/output unit 220 (step S362), the odor control unit 258 calculates a body center position of the subject and an abdominal width from the captured image (step S364).

Then, if the distance of shaking by the subject based on the body center position exceeds a fixed ratio of the abdominal width, the odor control unit 258 makes the odor production apparatus 30 produce an odor (steps S366 and S368). Since shaking of the body is thought to increase during nervousness, according to the sixth modified example, the subject's nervousness can be effectively alleviated by producing an odor when the subject is thought to be nervous.

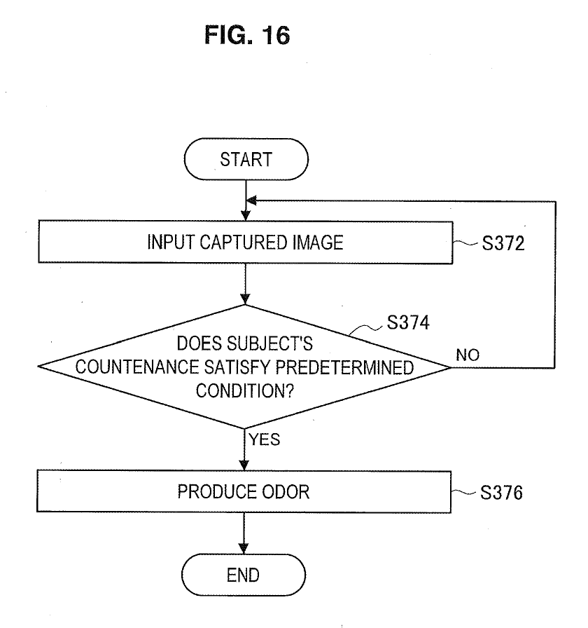

Further, the odor control unit 258 can also execute other controls based on the captured image of the subject. For example, as illustrated in FIG. 16, when a captured image of the subject is input to the input/output unit 220 (step S372), the odor control unit 258 determines whether the subject's countenance satisfies a predetermined condition (step S374). Since countenance is believed to stiffen when a person is nervous, the odor control unit 258 may determine whether the inclination angle of the subject's eyebrows or mouth exceed a set value as the predetermined condition.

If it is determined that the subject's countenance satisfies the predetermined condition, the odor control unit 258 makes the odor production apparatus 30 produce an odor (step S376). Thus, according to this configuration too, the subject's nervousness can be effectively alleviated by producing an odor when the subject is thought to be nervous.

### 3-7. Seventh Modified Example

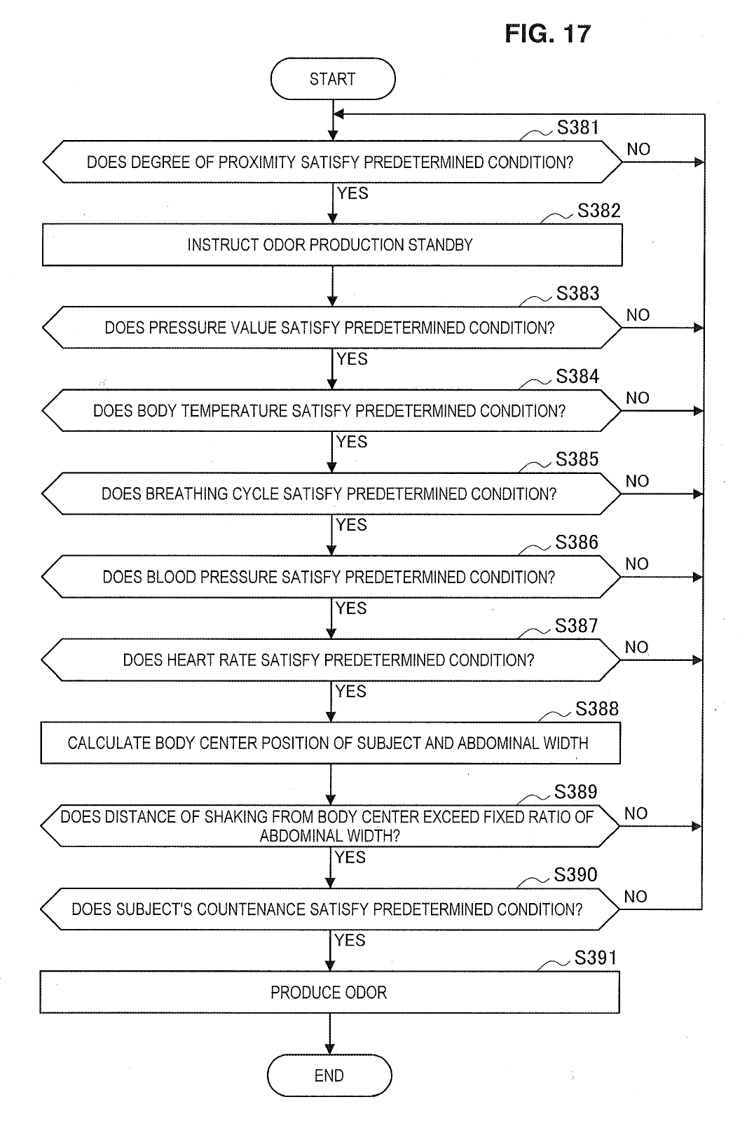

As a seventh embodiment, the above-described first embodiment and respective modified examples can be appropriately combined. A combined example of the first embodiment and respective modified examples will now be described with reference to FIG. 17.

FIG. 17 is a flowchart illustrating operation according to the seventh modified example. As illustrated in FIG. 17, first, as described in the first modified example, the odor control unit according to the seventh modified example determines whether a degree of proximity between the ultrasound probe 10 and the subject satisfies a predetermined condition (step S381). If the degree of proximity does satisfy the predetermined condition, the odor control unit instructs the odor production apparatus 30 to go on odor production standby (step S382).

Then, if each of step S383 to S387, S389, and S390 are determined in the affirmative, the odor control unit according to the seventh modified example makes the odor production apparatus 30 produce an odor (step S391). Specifically, if the pressure value (step S383), body temperature (step S384), breathing cycle (step S385), blood pressure (step S386), heart rate (step S387), shaking of the subject's body based on a calculation in step S388 (step S389), and subject's countenance satisfy the predetermined conditions, the odor control unit according to the seventh modified example makes the odor production apparatus 30 produce an odor (step S391).

### 4. SECOND EMBODIMENT

The first embodiment according to the present disclosure was described above. In the first embodiment, odor was described as an example of the environment perceived by the subject. However, in the second embodiment, a subject's nervousness can be alleviated by changing a variety of environments, such as music, video, humidity, and temperature. This will now be described in more detail.

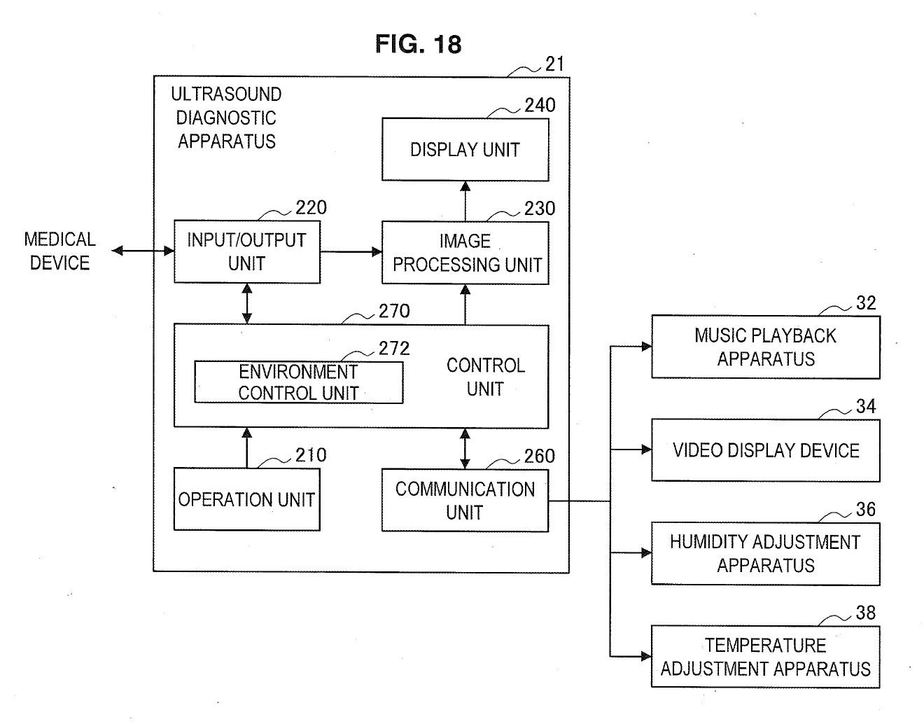

FIG. 18 is an explanatory diagram illustrating a configuration according to the second embodiment. As illustrated in FIG. 18, the environment control system according to the second embodiment includes an ultrasound diagnostic apparatus 21, a music playback apparatus 32, a video display device 34, a humidity adjustment apparatus 36, and a temperature adjustment apparatus 38.

The music playback apparatus 32 plays and outputs music based on instructions from the ultrasound diagnostic apparatus 21. The video display device 34 displays a video based on instructions from the ultrasound diagnostic apparatus 21. The video display device 34 can display the video on a display or project the video on a ceiling, a wall or the like. The humidity adjustment apparatus 36 adjusts the humidity by performing humidification or dehumidification based on instructions from the ultrasound diagnostic apparatus 21. The temperature adjustment apparatus 38 adjusts the temperature by operating a cooler or a heater based on instructions from the ultrasound diagnostic apparatus 21.

Further, as illustrated in FIG. 18, the ultrasound diagnostic apparatus 21 includes an operation unit 210, an input/output unit 220, an image processing unit 230, a display unit 240, a communication unit 260, and a control unit 270. Since the operation unit 210, the input/output unit 220, the image processing unit 230, the display unit 240, and the communication unit 260 are as described in the first embodiment, a description of those parts will be omitted here.

The control unit 270 controls the overall operation of the ultrasound diagnostic apparatus 21. Further, the control unit 270 according to the present embodiment has a function of an odor control unit 272. This environment control apparatus 272 controls the music playback apparatus 32, the video display device 34, the humidity adjustment apparatus 36, the temperature adjustment apparatus 38 and the like, based on information input into the input/output unit 220 from a medical device.

For example, if the information input from the medical device indicates that the medical device is being used or about to be used, the environment control unit 272 can make the music playback apparatus 32 play music that alleviates the subject's nervousness.

If the information input from the medical device indicates that the subject is in a nervous state, the environment control unit 272 can make the video display device 34 display a video that alleviates the subject's nervousness.

If the information input from the medical device indicates that the subject has a low body temperature, the environment control unit 272 can make the temperature adjustment apparatus 38 operate a heater. Further, if the information input from the medical device indicates that the subject's breath has a low humidity, the environment control unit 272 can make the humidity adjustment apparatus 36 perform humidification.

The environment control unit 272 may select the content of the music played by the music playback apparatus 32, the content of the video displayed on the video display device 34 and the like based on an attribute of the subject. For example, if the subject is an infant, the environment control unit 272 may select content such as music or cartoons for infants. In addition, the environment control unit 272 can select the content based on the subject's sex. Subject attributes can be acquired based on an input by the operator or from an electronic medical record.

As described above, according to the second embodiment, by changing a variety of environments perceived by the subject, the subject's nervousness can be expected to be alleviated even further.

### 5. THIRD EMBODIMENT

Next, a third embodiment of the present disclosure will be described. In the first and second embodiments of the present disclosure, the description was carried out mainly based on an ultrasound probe as an example of the medical device and an ultrasound diagnostic apparatus as an example of the environment control apparatus. However, like in the below-described third embodiment, the technology of the present disclosure can be applied to various medical devices and apparatuses.

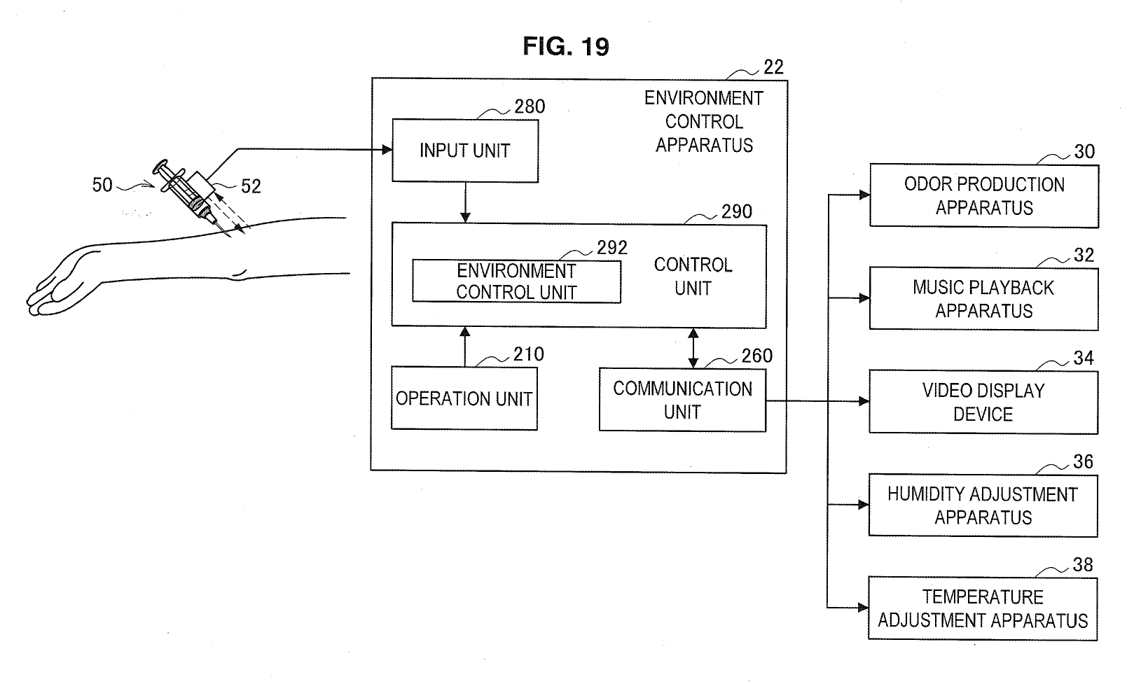

FIG. 19 is an explanatory diagram illustrating a configuration according to the third embodiment. As illustrated in FIG. 19, the environment control system according to the third embodiment includes an environment control apparatus 22, an odor production apparatus 30, a music playback apparatus 32, a video display device 34, a humidity adjustment apparatus 36, a temperature adjustment apparatus 38, and a syringe 50.

The syringe 50 includes a proximity sensor 52. The proximity sensor 52 detects whether the proximity sensor 52 is close to the subject. The proximity sensor 52 may be, for example, an infrared sensor that detects the time from transmission of infrared rays until reception of the reflected waves. Further, the proximity sensor 52 transmits a proximity detection result to the environment control apparatus 22 via a wire or wirelessly.

As illustrated in FIG. 19, the environment control apparatus 22 includes an operation unit 210, a communication unit 260, an input unit 280, and a control unit 290.

The operation unit 210 is a unit that lets an operator, such as a doctor or a medical technologist, operate the environment control apparatus 22. The operation unit 210 outputs signals to the control unit 290 based on operations made by the operator. A detection result by the proximity sensor 52 provided in the syringe 50 is input into the input unit 280. The communication unit 260 is an interface with the odor production apparatus 30, the music playback apparatus 32, the video display device 34, the humidity adjustment apparatus 36, and the temperature adjustment apparatus 38. This communication unit 260 may be connected to external devices via a wire or wirelessly.

The control unit 290 controls the overall operation of the environment control apparatus 22. Further, the control unit 290 according to the present embodiment has a function of an odor control unit 292. This environment control apparatus 292 controls the odor production apparatus 30, the music playback apparatus 32, the video display device 34, the humidity adjustment apparatus 36, the temperature adjustment apparatus 38 and the like, based on information input into the input unit 280 from the syringe 50. The determination criteria for the controls performed by the odor control unit 292 are as described in the first embodiment. The control content is as described in the second embodiment.

Thus, the technology according to the present disclosure can be applied to various medical devices and environments.

### 6. CONCLUSION

As described above, according to the respective embodiment of the present disclosure, alleviation of a subject's nervousness can be expected by changing a variety of environments perceived by the subject based on information input from a medical device.

It should be understood by those skilled in the art that various modifications, combinations, sub-combinations and alterations may occur depending on design requirements and other factors insofar as they are within the scope of the appended claims or the equivalents thereof.

For example, the environment control apparatus according to the present disclosure may control an environment perceived by the subject based on the sense of touch as the environment perceived by the subject. Specifically, the environment control apparatus may control generation of a low-frequency signal or physical vibration, pressing of a pressure point and the like, from which a massage effect can be obtained. By performing such an operation, the subject will relax, which should alleviate the subject's nervousness.

Further, the respective steps in the processing performed by the ultrasound diagnostic apparatus 20 according to the present disclosure do not have to be performed in chronological order according to the order illustrated in the flowcharts. For example, the respective steps in the processing performed by the ultrasound diagnostic apparatus 20 can be carried out in a different order to that illustrated in the flowcharts, or can be carried out in parallel.

In addition, a computer program can be created that makes hardware, such as a CPU, ROM, and RAM, in the ultrasound diagnostic apparatus 20 and the environment control apparatus 22, for example, realize functions equivalent to the respective constituents parts, such as the above-described ultrasound diagnostic apparatus 20 and the environment control apparatus 22. Still further, a storage medium on which such a computer program is stored is provided.

Additionally, the present technology may also be configured as below.
(1) An environment control apparatus including:

an input unit into which information from a medical device is input; and

an environment control unit configured to control an environment perceived by a subject based on the information input into the input unit.
(2) The environment control apparatus according to (1), wherein the environment control unit is configured to control the environment based on a usage state of the medical device indicated by the information input into the input unit.
(3) The environment control apparatus according to (2),

wherein a detection result by a pressure sensor is input into the input unit from the medical device, and

wherein the environment control unit is configured to determine whether the medical device and the subject are in contact from the detection result by the pressure sensor, and control the environment based on the contact determination result.
(4) The environment control apparatus according to (2) or (3),

wherein a detection result by a proximity sensor is input into the input unit from the medical device, and

wherein the environment control unit is configured to determine a proximity between the medical device and the subject from the detection result by the proximity sensor, and control the environment based on the proximity determination result.
(5) The environment control apparatus according to any one of (1) to (4), wherein the environment control unit is configured to control the environment based on a state of the subject indicated by the information input into the input unit.
(6) The environment control apparatus according to (5),

wherein a measurement result of body temperature is input into the input unit from the medical device, and

wherein the environment control unit is configured to control the environment based on the state of the subject indicated by the body temperature measurement result.
(7) The environment control apparatus according to (5) or (6), wherein a measurement result of blood pressure is input into the input unit from the medical device, and

wherein the environment control unit is configured to control the environment based on the state of the subject indicated by the blood pressure measurement result.
(8) The environment control apparatus according to any one of (5) to (7),

wherein a measurement result of breathing is input into the input unit from the medical device, and

wherein the environment control unit is configured to control the environment based on the state of the subject indicated by the breathing measurement result.
(9) The environment control apparatus according to any one of (5) to (8),

wherein a measurement result of pulse is input into the input unit from the medical device, and

wherein the environment control unit is configured to control the environment based on the state of the subject indicated by the pulse measurement result.
(10) The environment control apparatus according to any one of (1) to (9), wherein a captured image of the subject is further input into the input unit, and

wherein the environment control unit is configured to control the environment based on the captured image of the subject.
(11) The environment control apparatus according to (10), wherein the environment control unit is configured to determine a magnitude of shaking of the subject's body based on the captured image, and control the environment based on the magnitude of shaking of the body.
(12) The environment control apparatus according to (10) or (11), wherein the environment control unit is configured to determine a countenance of the subject based on the captured image, and control the environment based on the countenance determination result.
(13) The environment control apparatus according to any one of (1) to (12), wherein the environment control unit is configured to control production of an odor as the environment perceived by the subject.
(14) The environment control apparatus according to any one of (1) to (13), wherein the environment control unit is configured to control playback of music as the environment perceived by the subject.
(15) The environment control apparatus according to any one of (1) to (14), wherein the environment control unit is configured to control display of a video as the environment perceived by the subject.
(16) The environment control apparatus according to any one of (1) to (15), wherein the environment control unit is configured to control temperature as the environment perceived by the subject.
(17) The environment control apparatus according to any one of (1) to (16), wherein the environment control unit is configured to control humidity as the environment perceived by the subject.
(18) The environment control apparatus according to any one of (1) to (4), wherein the medical device is an ultrasound probe.
(19) An environment control method including:

receiving information from a medical device; and

controlling an environment perceived by a subject based on the information.
(20) A program for causing a computer to function as:

an input unit into which information from a medical device is input; and

an environment control unit configured to control an environment perceived by a subject based on the information input into the input unit.

The present disclosure contains subject matter related to that disclosed in Japanese Priority Patent Application JP 2012-049080 filed in the Japan Patent Office on Mar. 6, 2012, the entire content of which is hereby incorporated by reference.

What is claimed is:

1. An environment control apparatus comprising:
an input unit into which information from a medical device is input; and
an environment control unit configured to control an environment perceived by a subject based on the information input into the input unit.

2. The environment control apparatus according to claim 1, wherein the environment control unit is configured to control the environment based on a usage state of the medical device indicated by the information input into the input unit.

3. The environment control apparatus according to claim 2,
wherein a detection result by a pressure sensor is input into the input unit from the medical device, and
wherein the environment control unit is configured to determine whether the medical device and the subject are in contact from the detection result by the pressure sensor, and control the environment based on the contact determination result.

4. The environment control apparatus according to claim 2,
wherein a detection result by a proximity sensor is input into the input unit from the medical device, and
wherein the environment control unit is configured to determine a proximity between the medical device and the subject from the detection result by the proximity sensor, and control the environment based on the proximity determination result.

5. The environment control apparatus according to claim 1, wherein the environment control unit is configured to control the environment based on a state of the subject indicated by the information input into the input unit.

6. The environment control apparatus according to claim 5,
wherein a measurement result of body temperature is input into the input unit from the medical device, and
wherein the environment control unit is configured to control the environment based on the state of the subject indicated by the body temperature measurement result.

7. The environment control apparatus according to claim 5, wherein a measurement result of blood pressure is input into the input unit from the medical device, and
wherein the environment control unit is configured to control the environment based on the state of the subject indicated by the blood pressure measurement result.

8. The environment control apparatus according to claim 5,
wherein a measurement result of breathing is input into the input unit from the medical device, and
wherein the environment control unit is configured to control the environment based on the state of the subject indicated by the breathing measurement result.

9. The environment control apparatus according to claim 5,
wherein a measurement result of pulse is input into the input unit from the medical device, and
wherein the environment control unit is configured to control the environment based on the state of the subject indicated by the pulse measurement result.

10. The environment control apparatus according to claim 1, wherein a captured image of the subject is further input into the input unit, and
wherein the environment control unit is configured to control the environment based on the captured image of the subject.

11. The environment control apparatus according to claim 10, wherein the environment control unit is configured to determine a magnitude of shaking of the subject's body based on the captured image, and control the environment based on the magnitude of shaking of the body.

12. The environment control apparatus according to claim 10, wherein the environment control unit is configured to determine a countenance of the subject based on the captured image, and control the environment based on the countenance determination result.

13. The environment control apparatus according to claim 1, wherein the environment control unit is configured to control production of an odor as the environment perceived by the subject.

14. The environment control apparatus according to claim 1, wherein the environment control unit is configured to control playback of music as the environment perceived by the subject.

15. The environment control apparatus according to claim 1, wherein the environment control unit is configured to control display of a video as the environment perceived by the subject.

16. The environment control apparatus according to claim 1, wherein the environment control unit is configured to control temperature as the environment perceived by the subject.

17. The environment control apparatus according to claim 1, wherein the environment control unit is configured to control humidity as the environment perceived by the subject.

18. The environment control apparatus according to claim 1, wherein the medical device is an ultrasound probe.

19. An environment control method comprising:
receiving information from a medical device; and
controlling an environment perceived by a subject based on the information.

20. A program for causing a computer to function as:
an input unit into which information from a medical device is input; and
an environment control unit configured to control an environment perceived by a subject based on the information input into the input unit.
